(19) United States
(12) Patent Application Publication
(10) Pub. No.: US 2025/0064818 A1
(43) Pub. Date: Feb. 27, 2025

(54) DPP IV INHIBITOR FORMULATIONS

(71) Applicant: Boehringer Ingelheim International GmbH, Ingelheim am Rhein (DE)

(72) Inventors: Anja KOHLRAUSCH, Eberbach (DE); Patrick ROMER, Ochsenhausen (DE); Gerd SEIFFERT, Laupheim (DE)

(21) Appl. No.: 18/944,220

(22) Filed: Nov. 12, 2024

Related U.S. Application Data
(63) Continuation of application No. 17/319,325, filed on May 13, 2021, now Pat. No. 12,178,819.
Application No. 17/319,325 is a continuation of application No. 16/357,357, filed on Mar. 19, 2019, now Pat. No. 11,033,552.
Application No. 16/357,357 is a continuation of application No. 15/655,269, filed on Jul. 20, 2017, now abandoned.
Application No. 15/655,269 is a continuation of application No. 14/877,019, filed on Oct. 7, 2015, now abandoned.
Application No. 14/877,019 is a continuation of application No. 13/735,078, filed on Jan. 7, 2013, now abandoned.
Application No. 13/735,078 is a continuation of application No. 13/467,505, filed on May 9, 2012, now abandoned.
Application No. 13/467,505 is a continuation of application No. 13/230,133, filed on Sep. 12, 2011, now abandoned.
Application No. 13/230,133 is a continuation of application No. 11/744,701, filed on May 4, 2007, now abandoned.

(30) Foreign Application Priority Data
May 4, 2006 (EP) 06009201

(51) Int. Cl. A61K 31/522; A61K 9/00; A61K 9/20; A61K 9/28; A61K 31/517

(52) U.S. Cl. CPC A61K 31/522; A61K 9/2009; A61K 9/2013; A61K 9/2027; A61K 9/2054; A61K 9/2059; A61K 9/28; A61K 9/2813; A61K 9/2853; A61K 9/2866; A61K 9/2893; A61K 31/517; A61K 9/0053; A61K 9/2077

(57) ABSTRACT

The present invention relates to pharmaceutical compositions of DPP IV inhibitors with an amino group, their preparation and their use to treat diabetes mellitus.

### 1. FIELD OF THE INVENTION

The present invention relates to pharmaceutical compositions of selected DPP IV inhibitors, their preparation and their use to treat selected medical conditions.

### 2. DESCRIPTION OF THE PRIOR ART

The enzyme DPP-IV (dipeptidyl peptidase IV) also known as CD26 is a serine protease known to lead to the cleavage of a dipeptide from the N-terminal end of a number of proteins having at their N-terminal end a prolin or alanin residue. Due to this property DPP-IV inhibitors interfere with the plasma level of bioactive peptides including the peptide GLP-1 and are considered to be promising drugs for the treatment of diabetes mellitus.

### DETAILED DESCRIPTION OF THE INVENTION

In attempts to prepare pharmaceutical compositions of selected DPP-IV inhibitors it has been observed, that the DPP-IV inhibitors with a primary or secondary amino group show incompatibilities, degradation problems, or extraction problems with a number of customary excipients such as microcrystalline cellulose, sodium starch glycolate, croscarmellose sodium, tartaric acid, citric acid, glucose, fructose, saccharose, lactose, maltodextrines. Though the compounds themselves are very stable, they react with many excipients used in solid dosage forms and with impurities of excipients, especially in tight contact provided in tablets and at high excipient/drug ratios. The amino group appears to react with reducing sugars and with other reactive carbonyl groups and with carboxylic acid functional groups formed for example at the surface of microcrystalline cellulose by oxidation. These unforeseen difficulties are primarily observed in low dosage ranges which are required due to the surprising potency of the selected inhibitors. Thus, pharmaceutical compositions are required so solve these technical problems associated with the unexpected potency of selected DPP-IV inhibitor compounds.

A pharmaceutical composition according to the present invention is intended for the treatment of to achieve glycemic control in a type 1 or type 2 diabetes mellitus patient and comprises a DPP-IV inhibitor with an amino group, especially a free or primary amino group, as an active ingredient, a first and second diluent, a binder, a disintegrant and a lubricant. An additional disintegrant and an additional glidant are a further option. Additionally the compositions can be used to treat rheumatoid arthritis, obesity and osteoporosis as well as to support allograft transplantation.

Diluents suitable for a pharmaceutical composition according to the invention are cellulose powder, dibasic calciumphosphate anhydrous, dibasic calciumphosphate dihydrate, erythritol, low substituted hydroxypropyl cellulose, mannitol, pregelatinized starch or xylitol. Among those diluents mannitol and pregelatinized starch are preferred.

Diluents preferred as the second diluent are the above mentioned diluents pre-gelatinized starch and low-substituted hydroxypropylcellulose (L-HPC) which show additional binder properties.

Lubricants suitable for a pharmaceutical composition according to the invention are talc, polyethyleneglycol, calcium behenate, calcium stearate, hydrogenated castor oil or magnesium stearate. The preferred lubricant is magnesium stearate.

Binders suitable for a pharmaceutical composition according to the invention are copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, low-substituted hydroxypropylcellulose (L-HPC), copovidone and pregelatinized starch being preferred.

The above mentioned binders pregelatinized starch and L-HPC show additional diluent and disintegrant properties and can also be used as the second diluent or the disintegrant.

Disintegrants suitable for a pharmaceutical composition according to the present invention are corn starch, crospovidone, low-substituted hydroxypropylcellulose (L-HPC) or pregelatinized starch, corn starch being preferred.

As an optional glidant colloidal silicon dioxide can be used.

An exemplary composition according to the present invention comprises the diluent mannitol, pregelatinized starch as a diluent with additional binder properties, the binder copovidone, the disintegrant corn starch, and magnesium stearate as the lubricant.

Dosage forms prepared with a pharmaceutical compositions according to the present invention contain active ingredients in dosage ranges of 0.1-100 mg. Preferred dosages are 0.5 mg, 1 mg, 2.5 mg, 5 mg and 10 mg.

Typical pharmaceutical compositions comprise (% by weight)

| | |
|---|---|
| 0.5-20% | active ingredient |
| 40-88% | diluent 1, |
| 3-40% | diluent 2, |
| 1-5% | binder, |
| 5-15% | disintegrant, and |
| 0.1-4% | lubricant. |

Preferred pharmaceutical compositions comprise (% by weight)

| | |
|---|---|
| 0.5-7% | active ingredient |
| 50-75% | diluent 1, |
| 5-15% | diluent 2, |
| 2-4% | binder, |
| 8-12% | disintegrant, and |
| 0.5-2% | lubricant |

The pharmaceutical compositions according to the invention are intended for oral use and can be used in the dosage form of a capsule, a tablet or a film-coated tablet. Typically the film coat represents 2-4%, preferably 3% of the composition and comprises a film-forming agent, a plasticizer, a glidant and optionally one or more pigments. An exemplary coat composition may comprise hydroxypropylmethyl-cellulose (HPMC), polyethylene glycol (PEG), talc, titanium dioxide and optionally iron oxide.

Preferred active ingredients in the context of the present invention are DPP-IV inhibitors with a primary amino group and salts thereof such as any DPP-IV inhibitor and salt thereof defined by formula (I)

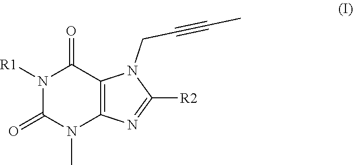

or formula (II)

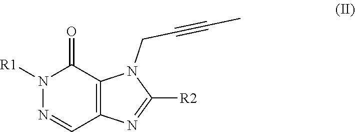

wherein R1 is ([1,5]naphthyridin-2-yl)methyl, (quinazolin-2-yl)methyl], (quinoxalin-6-yl)methyl, (4-Methyl-quinazolin-2-yl)methyl, 2-Cyano-benzyl, (3-Cyano-quinolin-2-yl)methyl, (3-Cyano-pyridin-2-yl)methyl, (4-Methyl-pyrimidin-2-yl)methyl, or (4,6-Dimethyl-pyrimidin-2-yl)methyl, and R2 is 3-(R)-amino-piperidin-1-yl, (2-amino-2-methyl-propyl)-methylamino or (2-(S)-amino-propyl)-methylamino.

Preferred DPP IV inhibitor compounds are the following compounds and salts thereof:
1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142):

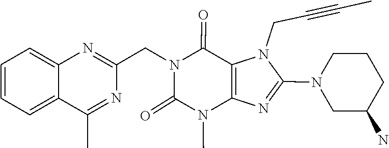

1-[([1,5]naphthyridin-2-yl)methyl]-3-methyl-7-(2-butyn-1-yl)-8-((R)-3-amino-piperidin-1-yl)-xanthine (compare WO 2004/018468, example 2(252)):

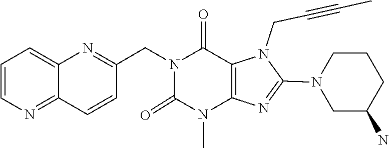

1-[(Quinazolin-2-yl)methyl]-3-methyl-7-(2-butyn-1-yl)-8-((R)-3-amino-piperidin-1-yl)-xanthine (compare WO 2004/018468, example 2(80)):

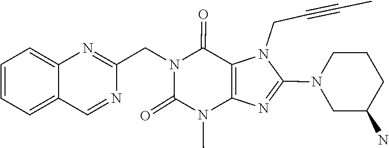

2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136):

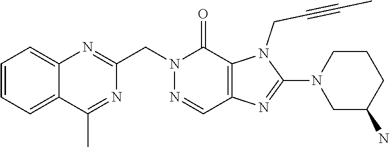

1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1)):

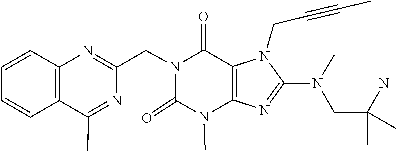

1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30)):

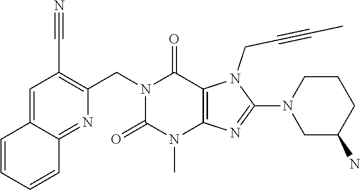

1-(2-Cyano-benzyl)-3-methyl-7-(2-butyn-1-yl)-8-((R)-3-amino-piperidin-1-yl)-xanthine (compare WO 2005/085246, example 1(39)):

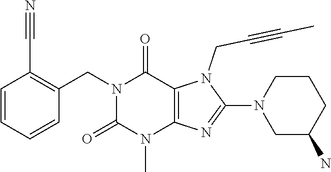

1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4)):

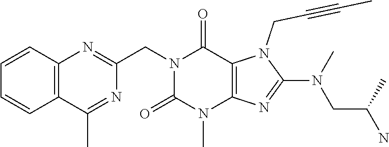

1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52)):

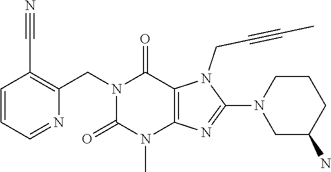

1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81)):

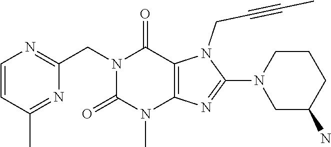

1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82)):

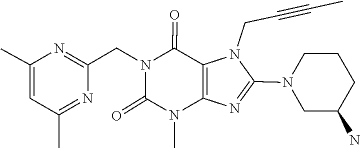

1-[(Quinoxalin-6-yl)methyl]-3-methyl-7-(2-butyn-1-yl)-8-((R)-3-amino-piperidin-1-yl)-xanthine (compare WO 2005/085246, example 1(83)):

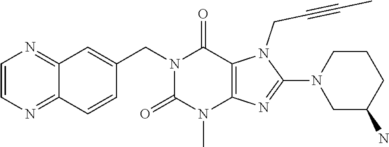

To prepare compositions according to the invention a granulate can be prepared by a wet granulation process. Alternative methods for granulation of active ingredient and excipients with a granulation liquid are fluid bed granulation or one-pot granulation.

In the wet granulation process the granulation liquid is a solvent such as water, ethanol, methanol, isopropanol, acetone, preferably purified water, and contains a binder such as copovidone. The solvent is a volatile component, which does not remain in the final product. The active ingredient and the other excipients with exception of the lubricant are premixed and granulated with the aqueous granulation liquid using a high shear granulator. The wet granulation step is followed by an optional wet sieving step, drying and dry sieving of the granules. For example a fluid bed dryer can then be used for drying.

The dried granules are sieved through an appropriate sieve. After addition of the other excipients with exception of the lubricant the mixture is blended in a suitable conventional blender such as a free fall blender followed by addition of the lubricant such as magnesium stearate and final blending in the blender.

Thus an exemplary wet granulation process for the preparation of a pharmaceutical composition according to the present invention comprises
a. dissolving a binder such as copovidone in a solvent such as purified water at ambient temperature to produce a granulation liquid;
b. blending a DPP-IV inhibitor, a diluent, and a disintegrant in a suitable mixer, to produce a pre-mix;
c. moistening the pre-mix with the granulation liquid and subsequently granulating the moistened pre-mix for example in a high shear mixer;
d. optionally sieving the granulated pre-mix through a sieve with a mesh size of at least 1.0 mm and preferably 3 mm;
e. drying the granulate at about 40-75° C. and preferably 55-65° C. inlet air temperature for example in a fluid bed dryer until the desired loss on drying value in the range of 1-5% is obtained;
f. delumping the dried granulate for example by sieving through a sieve with a mesh size of 0.6 mm-1.6 mm, preferably 1.0 mm; and
g. adding preferably sieved lubricant to the granulate for final blending for example in a cube mixer.

In an alternative process part of the exipients such as part of a disintegrant (e.g. corn starch) or a diluent (e.g. pregelatinized starch) or an additional disintegrant (crospovidone) can be added extragranular prior to final blending of step g.

In another alternative version of the process the granulate produced in steps a to e is produced in a one pot high shear granulation process and subsequent drying in a one pot granulator.

For the preparation of capsules the final blend is further filled into capsules.

For the preparation of tablets or tablet cores the final blend is further compressed into tablets of the target tablet core weight with appropriate size and crushing strength, using an appropriate tablet press.

For the preparation of film-coated tablets a coating suspension is prepared and the compressed tablet cores are coated with the coating suspension to a weight gain of about 2-4%, preferably about 3%, using a standard film coater. The film-coating solvent is a volatile component, which does not remain in the final product. To reduce the required amount of lubricant in the tablets it is an option to use an external lubrication system.

### EXAMPLES

### Example 1—Formulation for Direct Compression

An active DPP IV inhibitor ingredient with a primary amino group and all other excipients with exception of magnesium stearate are blended in a high shear blender. This pre-mix is sieved through a 1 mm sieve. After addition of magnesium stearate the pre-mix is blended in a free fall blender to produce the final blend. The final blend is compressed into tablets using a suitable tablet press. The following compositions can be obtained:

| Component | mg/tablet | %/tablet | mg/tablet | %/tablet |
|---|---|---|---|---|
| Active ingredient | 1.000 | 2.000 | 2.500 | 2.000 |
| Mannitol | 43.250 | 86.500 | 108.125 | 86.500 |
| Pregelatinized starch | 5.000 | 10.000 | 12.500 | 10.000 |
| Magnesium stearate | 0.750 | 1.500 | 1.875 | 1.500 |
| Total | 50.000 | 100.000 | 125.000 | 100.000 |
| Active ingredient | 5.000 | 2.000 | 10.000 | 2.000 |
| Mannitol | 216.250 | 86.500 | 432.500 | 86.500 |
| Pregelatinized starch | 25.000 | 10.000 | 50.000 | 10.000 |
| Magnesium stearate | 3.750 | 1.500 | 7.500 | 1.500 |
| Total | 250.000 | 100.000 | 500.000 | 100.000 |

### Example 2—Alternative Formulation for Direct Compression

An active DPP IV inhibitor ingredient with a primary amino group and all other excipients with exception of magnesium stearate are blended in a high shear blender. This pre-mix is sieved through a 1 mm sieve. After addition of magnesium stearate the pre-mix is blended in a free fall blender to produce the final blend. The final blend is compressed into tablets using a suitable tablet press. The following compositions can be obtained:

| Component | mg/tablet | %/tablet | mg/tablet | %/tablet |
|---|---|---|---|---|
| Active ingredient | 1.000 | 1.667 | 0.500 | 0.833 |
| Dibasic | 46.400 | 77.333 | 46.900 | 78.177 |
| calciumphosphate, | | | | |
| anhydrous | | | | |
| Low-substituted | 12.000 | 20.000 | 12.000 | 20.000 |
| hydroxypropylcellulose | | | | |
| Magnesium stearate | 0.600 | 1.000 | 0.600 | 1.000 |
| Total | 60.000 | 100.000 | 60.000 | 100.000 |
| Active ingredient | 10.000 | 1.667 | 10.000 | 2.222 |
| Dibasic | 464.000 | 77.333 | 344.000 | 76.788 |
| calciumphosphate, | | | | |
| anhydrous | | | | |
| Low-substituted | 120.000 | 20.000 | 90.000 | 20.000 |
| hydroxypropylcellulose | | | | |
| Magnesium stearate | 6.000 | 1.000 | 6.000 | 1.000 |
| Total | 600.000 | 100.000 | 450.000 | 100.000 |

### Example 3—Tablet Formulation

Copovidone is dissolved in purified water at ambient temperature to produce a granulation liquid. An active DPP IV inhibitor ingredient with a primary amino group, mannitol and part of the pregelatinized starch are blended in a suitable mixer, to produce a pre-mix. The pre-mix is moistened with the granulation liquid and subsequently granulated. The moist granulate is optionally sieved through a sieve with a mesh size of 1.6-3.0 mm. The granulate is dried at 55° C. in a suitable dryer to a residual moisture content corresponding to 2-5% loss on drying. The dried granulate is sieved through a sieve with a mesh size of 1.0 mm. The granulate is blended with part of the pregelatinized starch in a suitable mixer. Magnesium stearate is added to this blend after passing through a 1.0 mm sieve for delumping. Subsequently the final blend is produced by final blending in a suitable mixer and compressed into tablets. The following tablet composition can be obtained:

| Component | mg/tablet | %/tablet |
|---|---|---|
| Active ingredient | 10.000 | 1.667 |
| Pregelatinized starch | 210.000 | 35.000 |
| Mannitol | 236.000 | 39.333 |
| Copovidone | 18.000 | 3.000 |
| Total (granulate) | 474.000 | 79.000 |
| Pregelatinized starch | 120.000 | 20.000 |
| Magnesium stearate | 6.000 | 1.000 |
| Total | 600.000 | 100.000 |

### Example 4—Coated Tablet Formulation

Copovidone is dissolved in purified water at ambient temperature to produce a granulation liquid. An active DPP IV inhibitor ingredient with a primary amino group, mannitol, pregelatinized starch and corn starch are blended in a suitable mixer to produce the pre-mix. The pre-mix is moistened with the granulation liquid and subsequently granulated using a high shear mixer. The moist granulate is optionally sieved through a sieve with a mesh size of 1.6-3.0 mm. The granulate is dried at about 60° C. in a fluid bed dryer until a loss on the drying value of 2-4% is obtained. The Final Blend is compressed into tablet cores.

Hydroxypropyl methylcellulose, polyethylene glycol, talc, titanium dioxide and iron oxide are suspended in purified water in a suitable mixer at ambient temperature to produce a coating suspension. The tablet cores are coated with the coating suspension to a weight gain of about 3% to produce film-coated tablets. The following tablet compositions can be obtained:

| Component | mg | mg | mg | mg | mg |
|---|---|---|---|---|---|
| Active | 0.500 | 1.000 | 2.500 | 5.000 | 10.000 |
| ingredient | | | | | |
| Mannitol | 67.450 | 66.950 | 65.450 | 130.900 | 125.900 |
| Pregelatinized | 9.000 | 9.000 | 9.000 | 18.000 | 18.000 |
| starch | | | | | |
| Corn starch | 9.000 | 9.000 | 9.000 | 18.000 | 18.000 |
| Copovidone | 2.700 | 2.700 | 2.700 | 5.400 | 5.400 |
| Magnesium | 1.350 | 1.350 | 1.350 | 2.700 | 2.700 |
| stearate | | | | | |
| Total Mass | 90.000 | 90.000 | 90.000 | 180.000 | 180.000 |
| (tablet core) | | | | | |
| HPMC | 1.500 | 1.500 | 1.500 | 2.500 | 2.500 |
| PEG | 0.150 | 0.150 | 0.150 | 0.250 | 0.250 |
| Titanium | 0.750 | 0.750 | 0.750 | 1.250 | 1.250 |
| dioxide | | | | | |
| Talc | 0.525 | 0.525 | 0.525 | 0.875 | 0.875 |
| Iron oxide, | 0.075 | 0.075 | 0.075 | 0.125 | 0.125 |
| yellow | | | | | |
| Total Mass | 93.000 | 93.000 | 93.000 | 185.000 | 185.000 |
| (coated | | | | | |
| tablet) | | | | | |

### Example 5—Tablet Formulation

Copovidone is dissolved in purified water at ambient temperature to produce a granulation liquid. An active DPP IV inhibitor ingredient with a primary amino group, mannitol and pregelatinized starch are blended in a suitable mixer to produce a pre-mix. The pre-mix is moistened with the granulation liquid and subsequently granulated. The moist granulate is optionally sieved through a suitable sieve. The granulate is dried at about 50° C. in a suitable dryer until a loss on drying value of 3-5% is obtained. The dried granulate is sieved through a sieve with a mesh size of 1.0 mm.

Magnesium stearate is passed through a 1.0 mm sieve and added to the granulate. Subsequently the final blend is produced by final blending in a suitable blender and the final blend is compressed into tablets. The following tablet compositions can be obtained:

| Component | mg | mg | mg | mg | mg |
|---|---|---|---|---|---|
| Active ingredient | 0.500 | 1.000 | 2.500 | 5.000 | 10.000 |
| Mannitol | 27.500 | 27.000 | 67.500 | 135.000 | 130.000 |
| Pregelatinized starch | 20.000 | 20.000 | 50.000 | 100.000 | 100.000 |
| Copovidone | 1.500 | 1.500 | 3.750 | 7.500 | 7.500 |
| Magnesium stearate | 0.500 | 0.500 | 1.250 | 2.500 | 2.500 |
| Total tablet mass | 50.000 | 50.000 | 125.000 | 250.000 | 250.000 |

### Example 6—Tablet Formulation Variants

Copovidone is dissolved in purified water at ambient temperature to produce a granulation liquid. An active DPP IV inhibitor ingredient with a primary amino group and a part of mannitol, pregelatinized starch and corn starch are blended in a suitable mixer, to produce a pre-mix. The pre-mix is moistened with the granulation liquid and subsequently granulated. The moist granulate is sieved through a suitable sieve. The granulate is dried at about 60° C. inlet air temperature in a fluid bed dryer until a loss on drying value of 1-4% is obtained. The dried granulate is sieved through a sieve with a mesh size of 1.0 mm.

Magnesium stearate is passed through a sieve for delumping and added to the granulate. Additionally the remaining part of the exipients are added extragranular at this process step. Subsequently the final blend is produced by final blending in a suitable blender and compressed into tablet cores.

Hydroxypropyl methylcellulose, polyethylene glycol, talc, titanium dioxide and iron oxide are suspended in purified water in a suitable mixer at ambient temperature to produce a coating suspension. The tablet cores are coated with the coating suspension to a weight gain of about 3% to produce film-coated tablets. The following formulation variants can be obtained:

### Example 6.1—Formulation Variants With Extragranular Excipients

| Component | Formulation E | | Formulation F | |
|---|---|---|---|---|
| | mg/Tablet | %/Tablet | mg/Tablet | %/Tablet |
| Active ingredient | 1.000 | 1.111 | 1.000 | 1.111 |
| Mannitol | 23.300 | 25.889 | 66.950 | 74.389 |
| Pregelatinized starch | 4.500 | 5.000 | 4.500 | 5.000 |
| Corn starch | 4.500 | 5.000 | 4.500 | 5.000 |
| Copovidone | 1.350 | 1.500 | 2.700 | 3.000 |
| Total (granulate) | 34.650 | 38.500 | 79.650 | 88.500 |
| Corn starch | 4.500 | 5.000 | 4.500 | 5.000 |
| Pregelatinized starch | 4.500 | 5.000 | 4.500 | 5.000 |
| Mannitol | 45.000 | 50.000 | | |
| Magnesium stearate | 1.350 | 1.500 | 1.350 | 1.500 |
| Total (tablet core) | 90.000 | 100.000 | 90.000 | 100.000 |

### Example 6.2—Formulation Variants With Additional Extragranular Disintegrant

| Component | mg | mg | mg | mg | mg |
|---|---|---|---|---|---|
| Active ingredient | 0.500 | 1.000 | 2.500 | 5.000 | 10.000 |
| Mannitol | 67.450 | 66.950 | 65.450 | 130.900 | 125.900 |
| Pregelatinized starch | 9.000 | 9.000 | 9.000 | 18.000 | 18.000 |
| Corn starch | 9.000 | 9.000 | 9.000 | 18.000 | 18.000 |
| Copovidone | 2.700 | 2.700 | 2.700 | 5.400 | 5.400 |
| Total Mass | 88.650 | 88.650 | 88.650 | 177.300 | 177.300 |
| (granulate) | | | | | |
| Magnesium stearate | 1.350 | 1.350 | 1.350 | 2.700 | 2.700 |
| Crospovidone | 2.000 | 2.000 | 2.000 | 4.000 | 4.000 |
| Total Mass | 92.000 | 92.000 | 92.000 | 184.000 | 184.000 |
| (tablet core) | | | | | |
| HPMC | 1.500 | 1.500 | 1.500 | 2.500 | 2.500 |
| PEG | 0.150 | 0.150 | 0.150 | 0.250 | 0.250 |
| Titanium dioxide | 0.750 | 0.750 | 0.750 | 1.250 | 1.250 |
| Talc | 0.525 | 0.525 | 0.525 | 0.875 | 0.875 |
| Iron oxide, yellow | 0.075 | 0.075 | 0.075 | 0.125 | 0.125 |
| Total Mass | 95.000 | 95.000 | 95.000 | 189.000 | 189.000 |
| (coated tablet) | | | | | |

### Example 6.3—High Dose Formulations D

| Component | mg/tablet | %/tablet | mg/tablet | %/tablet |
|---|---|---|---|---|
| Active ingredient | 25.000 | 27.778 | 50.000 | 27.778 |
| Mannitol | 40.700 | 45.222 | 81.400 | 45.222 |
| Pregelatinized | 9.000 | 10.000 | 18.000 | 10.000 |
| starch | | | | |
| Corn starch | 9.000 | 10.000 | 18.000 | 10.000 |
| Copovidone | 2.700 | 3.000 | 5.400 | 3.000 |
| Total (granulate) | 86.400 | 96.000 | 172.800 | 96.000 |
| Crospovidone | 2.700 | 3.000 | 5.400 | 3.000 |
| Magnesium stearate | 0.900 | 1.000 | 1.800 | 1.000 |
| Total (tablet core) | 90.000 | 100.000 | 180.000 | 100.000 |
| Hydroxypropyl | 1.500 | 1.667 | 2.500 | 1.389 |
| methylcellulose | | | | |
| Polyethylene | 0.150 | 0.167 | 0.250 | 0.139 |
| glycol | | | | |
| Titanium dioxide | 0.750 | 0.833 | 1.250 | 0.694 |
| Talcum | 0.525 | 0.583 | 0.875 | 0.486 |
| Iron oxide yellow | 0.075 | 0.083 | 0.125 | 0.069 |
| Total (film-coated | 93.000 | 103.333 | 185.000 | 102.778 |
| tablet) | | | | |

What is claimed is:

1. A pharmaceutical composition comprising a compound of formula (I) 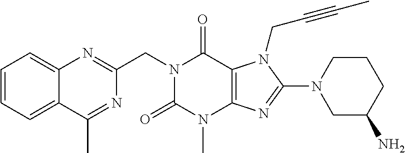 [SMILES: CC#CCn1c(N2CCC[C@@H](N)C2)nc2c1c(=O)n(Cc1nc(C)c3ccccc3n1)c(=O)n2C]
in an amount of 5 mg, or a salt thereof, a first diluent, a second diluent, a binder, a disintegrant and a lubricant, wherein the first and second diluents are independently cellulose powder, dibasic calcium phosphate anhydrous, dibasic calcium phosphate dihydrate, erythritol, low substituted hydroxypropyl cellulose, mannitol, pregelatinized starch, or xylitol,
wherein the pharmaceutical composition comprises:

| | |
|---|---|
| 0.5-20% | by weight of active ingredient |
| 40-88% | by weight of first diluent, |
| 3-40% | by weight of second diluent, |
| 1-5% | by weight of binder, |
| 5-15% | by weight of disintegrant, and |
| 0.1-4% | by weight of lubricant, |

and wherein pharmaceutical composition is in the dosage form of a tablet or a film-coated tablet.

2. The pharmaceutical composition of claim 1, wherein the first and second diluents are independently low substituted hydroxypropyl cellulose, mannitol, or pregelatinized starch.

3. The pharmaceutical composition of claim 1, wherein the lubricant is talc, polyethylene glycol, calcium behenate, calcium stearate, hydrogenated castor oil, or magnesium stearate.

4. The pharmaceutical composition of claim 1, wherein the binder is copovidone, hydroxypropyl methylcellulose (HPMC), hydroxypropylcellulose (HPC), or polyvinylpyrrolidone (Povidone).

5. The pharmaceutical composition of claim 1, wherein the disintegrant is corn starch.

6. The pharmaceutical composition of claim 1 further comprising an additional disintegrant.

7. The pharmaceutical composition of claim 6, wherein the additional disintegrant is crospovidone.

8. The pharmaceutical composition of claim 1 further comprising a glidant.

9. The pharmaceutical composition of claim 8, wherein the glidant is colloidal silicon dioxide.

10. The pharmaceutical composition of claim 1 comprising

| | |
|---|---|
| 0.5-7% | active ingredient |
| 50-75% | diluent 1, |
| 5-15% | diluent 2, |
| 2-4% | binder, |
| 8-12% | disintegrant, and |
| 0.5-2% | lubricant. |

11. The pharmaceutical composition of claim 1, wherein the film-coated tablet comprises 2-4% film coat.

12. The pharmaceutical composition of claim 11, wherein the film coat comprises a film-forming agent, a plasticizer, a glidant and optionally one or more pigments.

13. The pharmaceutical composition of claim 12, wherein the film coat comprises hydroxypropylmethylcellulose (HPMC), polyethylene glycol (PEG), talc, titanium dioxide and iron oxide.

14. A process for the preparation of a pharmaceutical composition according to claim 1 comprising
a. dissolving a binder in a solvent to produce a granulation liquid;
b. blending the compound of formula (I), a diluent, and a disintegrant to produce a pre-mix;
c. moistening the pre-mix with the granulation liquid and subsequently granulating the moistened pre-mix;
d. optionally sieving the granulated pre-mix through a sieve with a mesh size of at least 1.0 mm;
e. drying the granulate at about 40-75° C. until the desired loss on drying value in the range of 1-5% is obtained;
f. sieving the dried granulate through a sieve with a mesh size of at least 0.6 mm;
g. adding lubricant to the granulate for final blending.

15. The process according to claim 14 further comprising
h. compressing the final blend into tablet cores;
i. preparing a coating suspension;
j. coating the tablet cores with the coating suspension to a weight gain of about 2-4% to produce film-coated tablets.

16. The process according to claim 14, wherein part of the excipients are added extragranular prior to the final blending of step g.

17. The process according to claim 14, wherein the granulate produced in steps a-e is produced in a one pot high shear granulation process and subsequent drying in a one pot granulator.

18. The pharmaceutical composition of claim 1, wherein the first diluent is mannitol.

19. The pharmaceutical composition of claim 1, wherein the binder is copovidone, hydroxypropyl methylcellulose (HPMC), hydroxypropylcellulose (HPC), polyvinylpyrrolidon (povidone), pregelatinized starch, or low-substituted hydroxypropylcellulose (L-HPC).

20. The pharmaceutical composition of claim 1, wherein the disintegrant is corn starch, crospovidone, low-substituted hydroxypropylcellulose (L-HPC), or pregelatinized starch.

21. The pharmaceutical composition of claim 1, wherein the lubricant is talc, polyethyleneglycol, calcium behenate, calcium stearate, hydrogenated castor oil, or magnesium stearate.

22. The pharmaceutical composition of claim 1, wherein
the binder is copovidone, hydroxypropyl methylcellulose (HPMC), hydroxypropylcellulose (HPC), polyvinylpyrrolidon (povidone), pregelatinized starch, or low-substituted hydroxypropylcellulose (L-HPC); wherein
the disintegrant is corn starch, crospovidone, low-substituted hydroxypropylcellulose (L-HPC), or pregelatinized starch; and wherein
the lubricant is talc, polyethyleneglycol, calcium behenate, calcium stearate, hydrogenated castor oil, or magnesium stearate.

23. The pharmaceutical composition of claim 1, wherein the compound of formula (I) is present in an amount 0.5-7.0% based on the total weight of the compound of formula (I), first diluent, second diluent, binder, disintegrant and lubricant.
